(19) United States
(12) Patent Application Publication
(10) Pub. No.: US 2020/0163801 A1
(43) Pub. Date: May 28, 2020

(54) ELECTRONIC BANDAGE

(71) Applicant: FATRI (Xiamen) Technologies Co., Ltd., Xiamen, Fujian (CN)

(72) Inventor: Yongzhong NIE, Xiamen, Fujian (CN)

(73) Assignee: FATRI (XIAMEN) TECHNOLOGIES CO., LTD., Xiamen, Fujian (CN)

(21) Appl. No.: 16/617,809

(22) PCT Filed: Jun. 23, 2017
(86) PCT No.: PCT/CN2017/089786

§ 371 Date: Nov. 27, 2019

(30) Foreign Application Priority Data
May 31, 2017 (CN) 201710401599.3

(51) Int. Cl. A61F 13/00; A61N 1/04

(52) U.S. Cl. CPC A61F 13/00; A61F 2013/00119; A61N 1/0472; A61N 1/0468

(57) ABSTRACT

The embodiments of the present disclosure disclose an electronic bandage. The electronic bandage includes a fabric layer, an insulating layer and a conductive layer disposed in order from top to bottom; wherein the insulating layer is provided with a power source and a negative high voltage generator connected with each other, and the negative high voltage generator is further connected to the conductive layer; the conductive layer is provided with two or more support strips at intervals in length direction, the support strips protrude downward from the conductive layer, two or more microelectrodes are distributed on a lower surface of the conductive layer, and a height of a downward protruding portion of a support strip is greater than a height of a microelectrode.

### CROSS-REFERENCE TO RELATED APPLICATION

This application is a National Stage of International Application No. PCT/CN2017/089786, filed on Jun. 23, 2017, which claims priority to Chinese Patent Application No. 201710401599.3 filed on May 31, 2017, both of which are incorporated herein by reference in its entirety.

### TECHNICAL FIELD

The present disclosure relates to the field of medical technology, and more particularly to an electronic bandage.

### BACKGROUND

Currently, medical gauze is typically used in hospitals to bandage a wound of a patient after surgery. In order to prevent the bandaged wound from being infected, doctors need to disassemble the gauze and regularly apply anti-inflammatory drugs on a surface of the wound. However, frequent application of anti-inflammatory drugs on the surface of the wound can lead to drug resistance for human body.

### SUMMARY

Embodiments of the present disclosure provide an electronic bandage.

An embodiment of the present disclosure provides an electronic bandage including a fabric layer, an insulating layer and a conductive layer disposed in order from top to bottom; wherein the insulating layer is provided with a power source and a negative high voltage generator connected with each other, and the negative high voltage generator is further connected to the conductive layer; the conductive layer is provided with two or more support strips at intervals in length direction, the support strips protrude downward from the conductive layer, two or more microelectrodes are distributed on a lower surface of the conductive layer, and a height of a downward protruding portion of a support strip is greater than a height of a microelectrode.

In a possible implementation of the embodiment, wherein a distance S from an end of the downward protruding portion of the support strip to an end of the microelectrode satisfies S₀<S , wherein S₀ is a minimum distance between the downward protruding portion of the support strip and the end of the microelectrode that enables radiation of negative ions, and S₀ is calculated according to following formulas:

$$\begin{matrix}{S}_{0} = \frac{\partial {U}_{0}}{\partial {\vec{E}}_{0}} , & ( 1 ) \\ {U}_{0} = {\int }_{- {S}_{0}}^{0} {\vec{E}}_{0} {d}_{\vec{i}} , & ( 2 ) \\ {\vec{E} }_{0} = - ( \frac{\partial u}{\partial x} \vec{i} + \frac{\partial u}{\partial y} \vec{j} + \frac{\partial u}{\partial z} \vec{k} ) , & ( 3 )\end{matrix}$$

wherein i, j, k are a set of three-dimensional vectors, U₀ is an electrode escape voltage of an electric field formed between the microelectrode and human skin contacted by the support strip, and E₀ is an electric field strength of the electric field formed between the microelectrode and the human skin.

In a possible implementation of the embodiment, wherein the distance S from the end of the downward protruding portion of the support strip to the end of the microelectrode satisfies S₀<S<S₁, wherein S₀ is the minimum distance between the downward protruding portion of the support strip and the end of the microelectrode that enables radiation of negative ions, S₁ is a minimum distance between the downward protruding portion of the support strip and the end of the microelectrode that enables radiation of negative ions without air breakdown, and S₁ is calculated according to following formulas:

$$\begin{matrix}{S}_{1} = \frac{\partial {U}_{C}}{\partial {\vec{E}}_{0}} , & ( 4 ) \\ {U}_{C} = {\int }_{- {S}_{1}}^{0} {\vec{E}}_{0} {d}_{\vec{i}} , & ( 5 ) \\ {\vec{E} }_{0} = - ( \frac{\partial u}{\partial x} \vec{i} + \frac{\partial u}{\partial y} \vec{j} + \frac{\partial u}{\partial z} \vec{k} ) , & ( 6 )\end{matrix}$$

wherein i, j, k are a set of three-dimensional vectors, U_{C} is an air breakdown voltage of the electric field formed between the microelectrode and the human skin contacted by the support strip, and E₀ is the electric field strength of the electric field formed between the microelectrode and the human skin.

In a possible implementation of the embodiment, wherein the microelectrode comprises nano metal particles or nano conductive fibers.

In a possible implementation of the embodiment, wherein the two or more microelectrodes are evenly distributed on the lower surface of the conductive layer.

In a possible implementation of the embodiment, wherein the power source and the negative high voltage generator are disposed at a location in the insulating layer and adjacent to the fabric layer.

In a possible implementation of the embodiment, wherein the electronic bandage further comprises a lead wire connecting human body, one end of the lead wire is connected to the negative high voltage generator, and the other end of the lead wire passes through the fabric layer and extends outward.

In a possible implementation of the embodiment, wherein the electronic bandage further comprises a voltage regulator disposed in the insulating layer and a Bluetooth chip of the voltage regulator disposed in the insulating layer, the voltage regulator and the Bluetooth chip are both connected to the power source, and the voltage regulator is also connected to the Bluetooth chip and the negative high voltage generator, respectively.

In a possible implementation of the embodiment, wherein the electronic bandage further comprises a negative ion detector, the negative ion detector is disposed on a protruding portion of the support strip, and the negative ion detector is connected to the power source and the Bluetooth chip, respectively.

In a possible implementation of the embodiment, wherein an end of the downward protruding portion of the support strip has a chamfer shape.

In a possible implementation of the embodiment, wherein the support strip is an elastomer.

In a possible implementation of the embodiment, wherein the support strip is made of a biocompatible insulating polymer.

### BRIEF DESCRIPTION OF THE DRAWINGS

The present disclosure will be better understood from the following detailed description of non-limiting embodiments of the present disclosure with reference to the drawings in which like or similar reference numerals indicate like or similar features.

FIG. 1 is a schematic top view of a structure of an electronic bandage according to an embodiment of the present disclosure;

FIG. 2 is a schematic front view of t a structure of an electronic bandage according to an embodiment of the present disclosure;

FIG. 3 is a schematic cross-sectional view of a structure of an electronic bandage according to an embodiment of the present disclosure;

FIG. 4 is a schematic structural diagram of an electronic bandage according to an embodiment of the present disclosure;

FIG. 5 is a schematic structural diagram of an electronic bandage according to another embodiment of the present disclosure;

FIG. 6 is a schematic structural diagram of an electronic bandage according to another embodiment of the present disclosure;

### DETAILED DESCRIPTION

The features and exemplary embodiments of various aspects of the present disclosure will be described in detail below. In the following detailed description, numerous specific details are set forth in order to provide a thorough understanding of the present disclosure. However, it is obvious for those skilled in the art that the present disclosure can be practiced without some of the specific details. The following description of the embodiments is intended only to provide a better understanding of the present disclosure by showing examples of the present disclosure. The present disclosure is not limited to the specific configurations and algorithms set forth in the exemplary embodiments. Rather, the present disclosure can cover any modification, replacement and improvement of elements, components and algorithms, without departing from the scope of the present disclosure. In the accompanying drawings and the detailed description, the well-known structures and techniques are omitted in order not to obscure the present application.

The electronic bandage in the embodiment of the present disclosure may be applied to wound care. The usage of the electronic bandage can simplify the process for bandaging a wound, and can avoid the infection of the wound. Further, the usage of the electronic bandage can avoid the pain caused to a patient's skin when changing the bandage, and can improve the patient's experience.

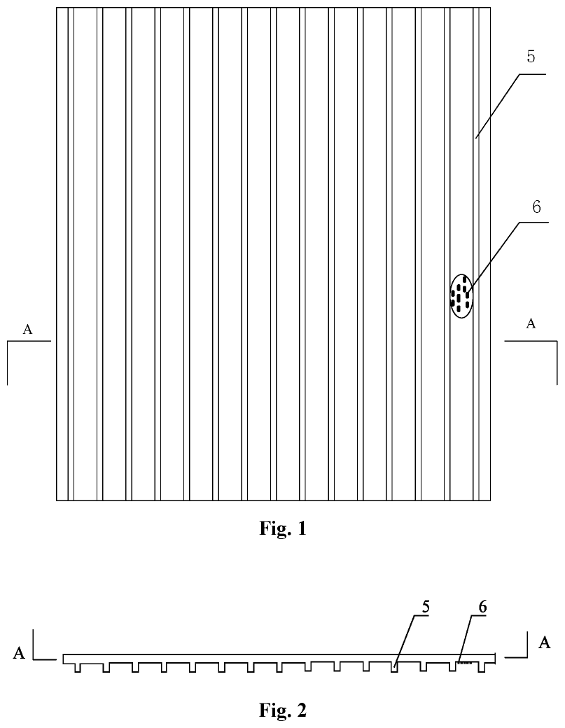

As shown in FIG. 1 to FIG. 3, the embodiments of the present disclosure provide an electronic bandage including a fabric layer 1, an insulating layer 2 and a conductive layer 3 disposed in order from top to bottom.

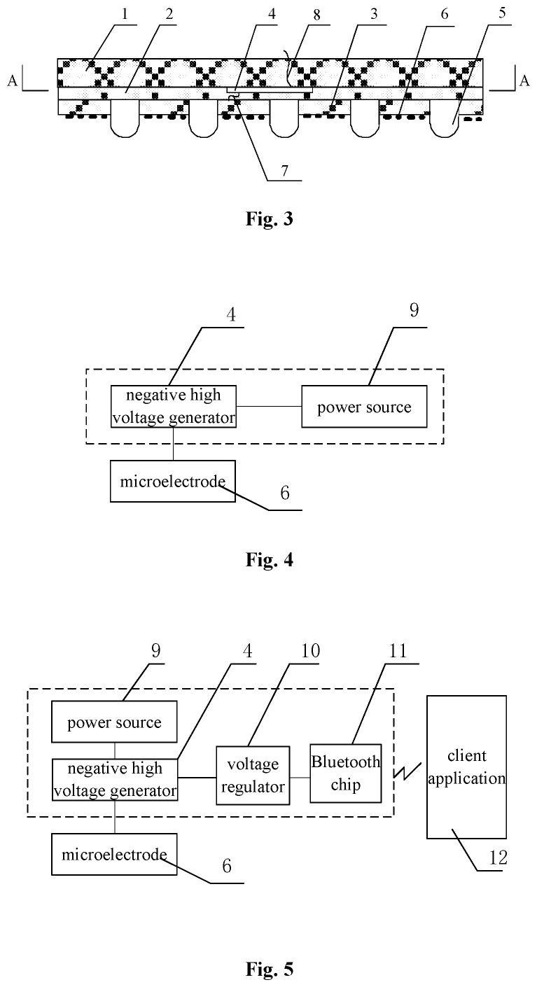

Referring to FIG. 4, the insulating layer 2 is provided with a power source 9 and a negative high voltage generator 4 connected with each other, and the negative high voltage generator 4 is further connected to the conductive layer 3. Wherein, the negative high voltage generator 4 is used to generate a negative high voltage capable of ionizing negative ions. Referring to FIG. 3, the negative high voltage can be applied to the microelectrodes via wires 7 provided in the insulating layer 2.

Referring to FIG. 3, the conductive layer 3 is provided with two or more support strips 5 at intervals in length direction, the support strips 5 protrude downward from the conductive layer 3, two or more microelectrodes 6 are distributed on a lower surface of the conductive layer 3, and a height of a downward protruding portion of a support strip 5 is greater than a height of a microelectrode 6. Wherein, the microelectrode 6 may include nano metal particles or nano conductive fibers. Of course, those skilled in the art can also use other microelectrodes 6 in nanometer size range, which is not limited in the embodiments of the present disclosure.

When the wound is bandaged using the electronic bandage in the embodiment of the present disclosure, the downward protruding portion of the support strip 5 of the electron bandage will contact the surface of the wounded skin and form an electric field between the microelectrode 6 and the surface of the wounded skin. After energizing, a negative high voltage generated by the negative high voltage generator 4 will be loaded onto the microelectrode 6 through the conductive layer 3. Under the function of the negative high voltage, the microelectrode 6 can ionize the air in the electric field to generate negative ions, and under the function of the electric field, the negative ions will migrate to the surface of the wounded skin. Since the negative ions have a bactericidal function, when the negative ions migrate to the surface of the wounded skin, the infection of the wound can be prevented, such that regular application of anti-inflammatory drugs on a surface of the wound is not needed, and drug resistance for human body will not happened.

In an embodiment, the two or more microelectrodes 6 are evenly distributed on the lower surface of the conductive layer 3. Therefore, the generation process of the negative ions may be more controllable.

Electric field strength formula may be E=U/d , wherein E is an electric field strength between two plates, U is a voltage between the two plates, and d is a distance between the two plates. In the embodiment of the present disclosure, in order to adjust the concentration of the negative ions ionized in the air between the microelectrode 6 and the surface of the wounded skin, the following two methods may be employed.

In the first method, the concentration of the negative ions ionized in the air between the microelectrode 6 and the surface of the wounded skin may be adjusted by adjusting a distance between the microelectrode 6 and the surface of the wounded skin, that is, a distance from an end of the downward protruding portion of the support strip 5 to an end of the microelectrode 6 of the electronic bandage.

For example, in order to enable radiation of negative ions in the air between the microelectrode 6 and the surface of the wounded skin, the distance S from the end of the downward protruding portion of the support strip 5 to the end of the microelectrode 6 may satisfy S₀<S, wherein S₀ is a minimum distance between the downward protruding portion of the support strip 5 and the end of the microelectrode 6 that enables radiation of the negative ions, and S₀ is calculated according to following formulas:

$$\begin{matrix}{S}_{0} = \frac{\partial {U}_{0}}{\partial {\vec{E}}_{0}} , & ( 1 ) \\ {U}_{0} = {\int }_{- {S}_{0}}^{0} {\vec{E}}_{0} {d}_{\vec{i}} , & ( 2 ) \\ {\vec{E} }_{0} = - ( \frac{\partial u}{\partial x} \vec{i} + \frac{\partial u}{\partial y} \vec{j} + \frac{\partial u}{\partial z} \vec{k} ) , & ( 3 )\end{matrix}$$

substituting formula (2) and formula (3) into formula (1), S₀ may be obtained, wherein i, j, k are a set of three-dimensional vectors, U₀ is an electrode escape voltage of an electric field formed between the microelectrode 6 and human skin contacted by the support strip 5, and E₀ is an electric field strength of the electric field formed between the microelectrode 6 and the human skin.

As another example, in order to enable radiation of negative ions in the air between the microelectrode 6 and the surface of the wounded skin and not to breakdown the air between the microelectrode 6 and the surface of the wounded skin, the distance S from the end of the downward protruding portion of the support strip 5 to the end of the microelectrode 6 may satisfy S₀<S<S₁, wherein S₀ is the minimum distance between the downward protruding portion of the support strip 5 and the end of the microelectrode 6 that enables radiation of negative ions, S₁ is a minimum distance between the downward protruding portion of the support strip 5 and the end of the microelectrode 6 that enables radiation of negative ions without air breakdown, and S₁ is calculated according to following formulas:

$$\begin{matrix}{S}_{1} = \frac{\partial {U}_{C}}{\partial {\vec{E}}_{0}} , & ( 4 ) \\ {U}_{C} = {\int }_{- {S}_{1}}^{0} {\vec{E}}_{0} {d}_{\vec{i}} , & ( 5 ) \\ {\vec{E} }_{0} = - ( \frac{\partial u}{\partial x} \vec{i} + \frac{\partial u}{\partial y} \vec{j} + \frac{\partial u}{\partial z} \vec{k} ) , & ( 6 )\end{matrix}$$

substituting formula (5) and formula (6) into formula (4), S₁ may be obtained, wherein i, j, k are a set of three-dimensional vectors, U_{C} is an air breakdown voltage of the electric field formed between the microelectrode 6 and the human skin contacted by the support strip 5, and E₀ is the electric field strength of the electric field formed between the microelectrode 6 and the human skin.

In the second method, the concentration of the negative ions ionized in the air between the microelectrode 6 and the surface of the wounded skin may be adjusted by adjusting a voltage between the microelectrode 6 and the surface of the wounded skin, that is, a negative high voltage loaded onto the microelectrode 6 by the negative high voltage generator 4.

For example, a voltage regulator 10 may be disposed in the insulating layer 2. The concentration of the negative ions ionized in the air between the microelectrode 6 and the surface of the wounded skin may be adjusted by adjusting the negative high voltage generated by the negative high voltage generator 4 via the voltage regulator 10.

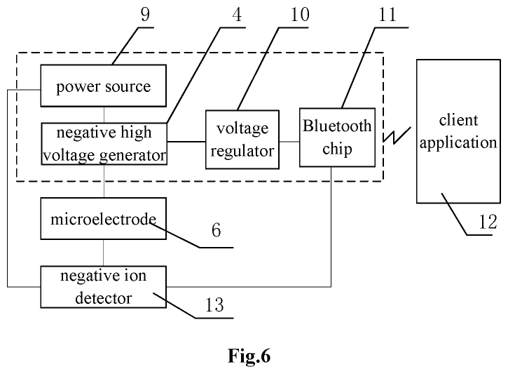

As another example, referring to FIG. 5, the electronic bandage may further include a voltage regulator 10 disposed in the insulating layer 2 and a Bluetooth chip 11 of the voltage regulator 10 disposed in the insulating layer 2, the voltage regulator 10 and the Bluetooth chip 11 may be both connected to the power source 9, and the voltage regulator 10 may be also connected to the Bluetooth chip 11 and the negative high voltage generator 4, respectively.

Wherein, the Bluetooth chip 11 may be used to pair with a user's mobile device. After the pairing is successful, the user may issue a user command of voltage increase or voltage decrease to the voltage regulator 10 of the electronic bandage via an associated client application 12. The voltage regulator 10 may receive the user command through the Bluetooth chip 11 in the electronic bandage, and accordingly adjust the negative high voltage generated by the negative high voltage generator 4, such that the concentration of the negative ions ionized in the air between the microelectrode 6 and the surface of the wounded skin may be adjusted.

Further, referring to FIG. 6, the electronic bandage may further include a negative ion detector 13, the negative ion detector 13 may be disposed on a protruding portion of the support strip 5, and the negative ion detector 13 may be connected to the power source 9 and the Bluetooth chip 11, respectively. After the Bluetooth chip 11 is paired with the user's mobile device, the associated client application 12 may receive the concentration of the negative ions detected by the negative ion detector 13. Therefore, the user may issue the user command of voltage increase or voltage decrease.

In order to calculate the concentration of the negative ions ionized in the air between the microelectrode 6 and the surface of the wounded skin, it can be assumed that each oxygen atom acquires a free electron and becomes a negative oxygen ion, and the concentration N of the negative ions may be calculated as N=I/q, wherein, I is a current between the microelectrode 6 and the surface of the wounded skin, and N is integer.

According to an embodiment of the present disclosure, in the electric field between the microelectrode 6 and the surface of the wounded skin, for the negative high voltage on the microelectrode 6, the potential at infinity is zero. In order to make the potential of the surface of the wounded skin as zero to increase the concentration of the negative ions ionized in the air between the microelectrode 6 and the surface of the wounded skin, the electronic bandage may further include a lead wire 8 connecting human body, one end of the lead wire 8 may be connected to the negative high voltage generator 4, and the other end of the lead wire 8 may pass through the fabric layer 1 and extend outward.

In an embodiment of the present disclosure, in order to optimize the structure of the electronic bandage and improve the breakdown strength of the insulating layer 2, the power source 9 and the negative high voltage generator 4 are disposed at a location in the insulating layer 2 and adjacent to the fabric layer 1.

Further, to improve the contact between the electronic bandage and the surface of the wounded skin, the support strip 5 may be an elastomer, the end of the downward protruding portion of the support strip 5 may have a chamfer shape, and the support strip 5 may be made of a biocompatible insulating polymer.

It should be noted that, in the embodiment of the present disclosure, the microelectrode 6 may also generate ozone while ionizing the negative ions in the air between the microelectrode 6 and the surface of the wounded skin. Since ozone may have a function of sterilizing and purifying the air, the bactericidal function of the electron bandage may be improved.

Example embodiments will now be described more fully with reference to the accompanying drawings. However, the example embodiments can be embodied in a variety of forms and should not be construed as being limited to the embodiments set forth herein; rather, these embodiments are provided to make the embodiments of the present disclosure more comprehensive and complete, and illustrate the idea of the embodiments to those skilled in the art. In the accompanying drawings, the thickness of the regions and layers may be exaggerated for clarity. The same reference numerals in the drawings denote the same or similar structures, and thus their detailed description will be omitted.

It should be clear that the embodiments in this specification are described in a progressive manner, and the same or similar parts in each embodiment may be referred to each other, and each embodiment focuses on the difference from other embodiments. For device embodiments, relevant parts can be referred to the description of the method embodiments. The embodiments of the present disclosure are not limited to the specific steps and structures described above and shown in the drawings. Those skilled in the art can make various changes, modifications and additions or changing the order between steps after understanding the spirit of the embodiments of the present disclosure. Also, for the sake of brevity, detailed descriptions of known methods and techniques are omitted here.

The embodiments of the present disclosure may be implemented in other specific forms without departing from its spirit and essential characteristics. For example, the algorithms described in the specific embodiments may be modified without system architecture departing from the basic spirit of the embodiments of the present disclosure. As such, the embodiments are to be considered in all respects as illustrative and not restrictive, and the scope of the embodiments of the disclosure is defined by the appended claims rather than the foregoing descriptions. All changes that are defined in the meaning and the equivalents of the claims come within the scope of the embodiments of the disclosure.

What is claimed is:

1. An electronic bandage comprising a fabric layer, an insulating layer and a conductive layer disposed in order from top to bottom, wherein:
the insulating layer is provided with a power source and a negative high voltage generator connected with each other, and the negative high voltage generator is further connected to the conductive layer;
the conductive layer is provided with two or more support strips at intervals in length direction, the support strips protrude downward from the conductive layer, two or more microelectrodes are distributed on a lower surface of the conductive layer, and a height of a downward protruding portion of a support strip is greater than a height of a microelectrode.

2. The electronic bandage according to claim 1, wherein a distance S from an end of the downward protruding portion of the support strip to an end of the microelectrode satisfies S₀<S, wherein S₀ is a minimum distance between the downward protruding portion of the support strip and the end of the microelectrode that enables radiation of negative ions, and S₀ is calculated according to following formulas:
$$\begin{matrix}{S}_{0} = \frac{\partial {U}_{0}}{\partial {\vec{E}}_{0}} , \\ {U}_{0} = {\int }_{- {S}_{0}}^{0} {\vec{E}}_{0} {d}_{\vec{i}} , \\ {\vec{E} }_{0} = - ( \frac{\partial u}{\partial x} \vec{i} + \frac{\partial u}{\partial y} \vec{j} + \frac{\partial u}{\partial z} \vec{k} ) ,\end{matrix}$$
wherein i, j, k are a set of three-dimensional vectors, U₀ is an electrode escape voltage of an electric field formed between the microelectrode and human skin contacted by the support strip, and E₀ is an electric field strength of the electric field formed between the microelectrode and the human skin.

3. The electronic bandage according to claim 2, wherein the distance S from the end of the downward protruding portion of the support strip to the end of the microelectrode satisfies S₀<S<S₁, wherein S₀ is the minimum distance between the downward protruding portion of the support strip and the end of the microelectrode that enables radiation of negative ions, S₁ is a minimum distance between the downward protruding portion of the support strip and the end of the microelectrode that enables radiation of negative ions without air breakdown, and S₁ is calculated according to following formulas:
$$\begin{matrix}{S}_{1} = \frac{\partial {U}_{C}}{\partial {\vec{E}}_{0}} , \\ {U}_{C} = {\int }_{- {S}_{1}}^{0} {\vec{E}}_{0} {d}_{\vec{i}} , \\ {\vec{E} }_{0} = - ( \frac{\partial u}{\partial x} \vec{i} + \frac{\partial u}{\partial y} \vec{j} + \frac{\partial u}{\partial z} \vec{k} ) ,\end{matrix}$$
wherein i, j, k are a set of three-dimensional vectors, U_{C} is an air breakdown voltage of the electric field formed between the microelectrode and the human skin contacted by the support strip, and E₀ is the electric field strength of the electric field formed between the microelectrode and the human skin.

4. The electronic bandage according to claim 1, wherein the microelectrode comprises nano metal particles or nano conductive fibers.

5. The electronic bandage according to claim 1, wherein the two or more microelectrodes are evenly distributed on the lower surface of the conductive layer.

6. The electronic bandage according to claim 1, wherein the power source and the negative high voltage generator are disposed at a location in the insulating layer and adjacent to the fabric layer.

7. The electronic bandage according to claim 1, wherein the electronic bandage further comprises a lead wire connecting human body, one end of the lead wire is connected to the negative high voltage generator, and the other end of the lead wire passes through the fabric layer and extends outward.

8. The electronic bandage according to claim 1, wherein the electronic bandage further comprises a voltage regulator disposed in the insulating layer and a Bluetooth chip of the voltage regulator disposed in the insulating layer, the voltage regulator and the Bluetooth chip are both connected to the power source, and the voltage regulator is also connected to the Bluetooth chip and the negative high voltage generator, respectively.

9. The electronic bandage according to claim 1, wherein the electronic bandage further comprises a negative ion detector, the negative ion detector is disposed on a protruding portion of the support strip, and the negative ion detector is connected to the power source and the Bluetooth chip, respectively.

10. The electronic bandage according to claim 1, wherein an end of the downward protruding portion of the support strip has a chamfer shape.

11. The electronic bandage according to claim 1, wherein the support strip is an elastomer.

12. The electronic bandage according to claim 1, wherein the support strip is made of a biocompatible insulating polymer.
